(19) United States
(12) Patent Application Publication
(10) Pub. No.: US 2025/0341082 A1
(43) Pub. Date: Nov. 6, 2025

(54) BEDPAN WASHER SINK APPARATUS

(71) Applicant: Spencer Todd Hughes, Coffeeville, MS (US)

(72) Inventor: Spencer Todd Hughes, Coffeeville, MS (US)

(21) Appl. No.: 19/197,165

(22) Filed: May 2, 2025

Related U.S. Application Data
(60) Provisional application No. 63/641,553, filed on May 2, 2024.
(60) Provisional application No. 63/709,852, filed on Oct. 21, 2024.

(51) Int. Cl. E03C 1/182; A61L 2/18

(52) U.S. Cl. CPC E03C 1/182; A61L 2/18; A61L 2202/121; A61L 2202/122; A61L 2202/123; A61L 2202/15; A61L 2202/17

(57) ABSTRACT

A bedpan washer sink can include a container section mounted on a backing plate. The container section defines an interior area for receiving and containing at least a portion of a bedpan washing wand therein. An opening is formed in the base panel that can allow liquid collected in the container section to drain from the container section. A hook can be mounted on the backing plate to support the hose of the bedpan washing wand. A compartment can be mounted on the backing plate above the container section, and a sanitizing device can be positioned in the compartment. The sanitizing device can be a bottle containing a sanitizing liquid having a pump dispenser. Sanitizing liquid dispensed from the bottle falls into the container section to sanitize the washing wand positioned therein.

### CROSS REFERENCE TO RELATED APPLICATIONS

This application claims priority to U.S. Provisional Patent Application No. 63/641,553, filed May 2, 2024, and U.S. Provisional Patent Application No. 63/709,852, filed Oct. 21, 2024. Both U.S. Provisional Patent Application No. 63/641,553 and U.S. Provisional Patent Application No. 63/709,852 are incorporated herein by reference.

### TECHNICAL FIELD

The present invention relates to plumbing fixtures. An embodiment of the invention comprises a bedpan washer sink

### BACKGROUND

U.S. Pat. No. 3,895,401 discloses a drain structure and is incorporated herein by reference. U.S. Pat. No. 4,109,939 discloses a device for attaching a fluid carrying hose to a receptacle drain and is incorporated herein by reference. U.S. Pat. No. 4,784,184 discloses an elastic drip silencer and funnel and is incorporated herein by reference. U.S. Pat. No. 5,690,138 discloses a receptacle for a dripping spigot and is incorporated herein by reference. U.S. Pat. No. 6,845,784 discloses a fire sprinkler water catching apparatus and is incorporated herein by reference. U.S. Pat. No. 7,992,586 discloses a receptacle for catching and retaining unwanted liquids and is incorporated herein by reference. U.S. Pat. No. 8,430,138 discloses a funnel and is incorporated herein by reference. U.S. Pat. No. 9,637,904 discloses a multi-spray bidet and is incorporated herein by reference. U.S. Patent Application Publication No. 2005/0060833 discloses a cleaning wand with multiple cleaning heads for use with an all surface cleaning apparatus and is incorporated herein by reference. U.S. Patent Application Publication No. 2011/0094603 discloses a residue collector and is incorporated herein by reference.

### SUMMARY

Cleaning instruments known as bedpan washing wands are commonly used in bathrooms of hospitals and assisted living centers where bedpans are common. After they are used to clean a bedpan, they are often hung on a hanger or hook on the bathroom wall where they are prone to leak water onto the floor, resulting in a dangerous and/or unsanitary hazard. Accordingly, one object of the present invention is to provide a means for conveniently storing a bedpan washing wand that can capture and collect water leaking from the wand. Another object of the present invention is to help prevent leaking water from contacting the floor, wall and/or other surrounding structures. Another object of the present invention is to provide an apparatus that can be installed in bathrooms in which bedpan washing wands are used that helps to maintain the bedpan washing wand and the bathroom generally in a sanitary condition. These and other objectives of the invention can be achieved in various embodiments of the invention disclosed herein.

An embodiment of the invention comprises an apparatus adapted for receiving and storing a moveable liquid conveying device, such as a bedpan washing wand.

One embodiment of the invention comprises a bedpan washer sink adapted for receiving and storing a bedpan washing wand. The sink comprises a container section for receiving the bedpan washing wand, an outlet section positioned at the bottom of the container section for evacuating liquid from the container section, and a planar section attached to the container section for mounting on a wall.

According to an embodiment of the invention, a tube is connected at one end to the outlet section and the other end of the tube is connected to a sanitary sewer, flush valve, or flushometer. Water and/or other liquids that typically leak from the bedpan washing wand are captured and collected in the container section and evacuated through the outlet section into the tube connected to the sanitary sewer.

According to an embodiment of the invention, the bed pan sink can be sold and installed as is, and can be installed in a non-invasive manner.

According to an embodiment of the invention, the bedpan sink can be connected to trap primer tail piece below the flush valve with vertical mounted swing check valve.

According to an embodiment of the invention, the bedpan sink can be made of 304 stainless steel polished plates.

Another embodiment of the invention comprises a method of installing the bedpan washer sink. Another embodiment of the invention comprises a method of using the bedpan washer sink.

Another embodiment of the invention comprises a plumbing fixture kit comprising a bedpan washing wand and a bedpan sink adapted to receive and store the bedpan washing wand when not in use. The sink can be comprised of a container section for receiving the bedpan washing wand, an outlet section positioned at the bottom of the container section for evacuating liquid from the container section, and a planar section attached to a back panel of the container section for mounting on a wall. A tube can be connected at one end to the outlet section and the opposite end can be connected to a sanitary sewer, flush valve, or flushometer. Water and other liquid that typically leaks from the bedpan washing wand is captured and collected in the container section and evacuated through the outlet section into the tube connected to the sanitary sewer.

Another embodiment of the invention comprises a bedpan washer sink with a sanitizing assembly for cleaning and/or sanitizing the washing wand after use. The washing wand can become dirty from being used to wash out dirty bedpans. As such, the sanitizing assembly helps maintain the washing wand in a clean and sanitary condition.

According to an embodiment of the invention, the bedpan washer sink apparatus can be installed in the bathrooms of healthcare facilities having a bedpan washer or similar cleaning instrument. The bedpan washer sink apparatus can be included in plumbing specifications for said bathrooms similar to a toilet and/or water fountain.

Another embodiment of the invention comprises bedpan washer sink apparatus comprising a substantially flat backing plate, and a container section comprising at least one side panel attached to a base panel and the backing plate. The container section defines an interior area for receiving and containing at least a portion of a bedpan washing wand therein. An opening is formed in the base panel that can allow liquid collected in the container section to drain from the container section.

According to an embodiment of the invention, the container section comprises two opposed side panels attached to the base panel and the backing plate, and a face panel attached to the two opposed side panels.

According to an embodiment of the invention, the container section can have the shape of a truncated pyramid or trapezoidal prism.

According to an embodiment of the invention, a plurality of openings can be formed in the backing plate to receive fasteners therethrough. The fasteners can be wall screws or other suitable fasteners.

According to an embodiment of the invention, a pipe coupling is attached around the opening formed in the base panel. A check valve can be connected to the pipe coupling.

According to an embodiment of the invention, at least one hook is attached to the backing plate to support a hose of the bedpan washer wand.

According to an embodiment of the invention, the bedpan washer sink apparatus can include a sanitizing device containing a sanitizing liquid, and a support member can be attached to the backing plate above the container section to support the sanitizing device thereon.

According to an embodiment of the invention, the support member can be a compartment comprising first and second opposed side panels attached to the backing plate, a base panel attached to the first and second opposed side panels and the backing plate, and a face panel attached to the first and second opposed side panels and the base panel. A drip chute can be positioned between the compartment and the container section.

Another embodiment of the invention comprises a bedpan washer sink kit comprising a backing plate having a plurality of openings formed therein, and a container section comprising at least one side panel attached to a base panel and the backing plate. The base panel has an opening formed therein for allowing liquid collected in the container section to drain from the container section. The kit includes a plurality of fasteners for positioning through the openings formed in the backing plate whereby the backing plate can be attached to a wall. The fasteners can be wall screws or other suitable fasteners.

According to an embodiment of the invention, a pipe coupling can be attached around the opening formed in the base panel of the container section and a check valve can be operatively connected to the pipe coupling.

According to another embodiment of the invention, the kit can include a tubular drain line. One end of the drain line can be operatively connected to the check valve, and the other end of the drain line can be operatively connected to a trap primer tail piece.

According to another embodiment of the invention, the kit includes a bedpan washing wand, and at least a portion of the bedpan washing wand is received and contained within the container section.

According to another embodiment of the invention, the bedpan washing wand includes a hose, and the bedpan washer sink apparatus includes at least one hook mounted on the backing plate for supporting the hose thereon.

According to another embodiment of the invention, a support member can be mounted on the backing plate above the container section, and a bottle containing a sanitizing liquid is positioned on the support member. The bottle can include a pump dispenser for dispensing the sanitizing liquid from the bottle. Sanitizing liquid exiting the pump dispenser falls into the container section.

Another embodiment of the invention comprises a method for sanitizing a bedpan washer wand and maintaining the bedpan washer wand in a sanitary condition. The method includes providing a bedpan washer sink apparatus comprising a substantially flat backing plate having a plurality of openings formed therein, and a container section comprising at least one side panel connected to a base panel and the backing plate. The base panel has an opening formed therein for allowing liquid collected in the container section to drain from the container section, an on/off valve operatively connected to the opening formed in the base panel, and a sanitizing device containing a sanitizing liquid. The backing plate is positioned against a wall and fasteners, such as wall screws, are inserted through the openings formed in the backing plate and into the wall whereby the backing plate is attached to the wall. A bedpan washer wand for cleaning items, such as bedpans, is provided. The bedpan washer wand has a terminal end from which liquid is dispensed. When the bedpan washer wand is not being used to clean, the bedpan washer wand is positioned in the container section of the bedpan washer sink apparatus. The on/off valve is closed (turned to the “off” position) thereby preventing evacuation of liquid through the opening in the base panel of the container section. Sanitizing liquid is dispensed from the sanitizing device into the container section. After a desired amount of time, the on/off valve can be opened (turned to “on” position) to allow liquid collected in the container section to exit the container section.

According to an embodiment of the invention, a drain line can be operatively connected to the bedpan washer sink apparatus and a toilet.

According to another embodiment of the invention, the sanitizing device can comprise a bottle containing a sanitizing liquid and having a pump dispenser for dispensing the sanitizing liquid from the bottle. A support member can be mounted on the backing plate above the container section, and the bottle can be positioned on the support member. Sanitizing liquid exiting the pump dispenser of the bottle falls into the container section.

### BRIEF DESCRIPTION OF THE DRAWINGS

FIG. 1 is a front view of a bedpan washer sink according to an embodiment of the invention;

FIG. 2 is a perspective view of the bedpan washer sink of FIG. 1;

FIG. 3 is a side view of the bedpan washer sink of FIG. 1;

FIG. 4 is an environmental view of the bedpan washer sink of FIG. 1;

FIG. 5 is another environmental view of the bedpan sink of FIG. 1;

FIG. 6 is a side view of a bedpan sink according to another embodiment of the invention; and

FIG. 7 is an environmental view of the bedpan sink of FIG. 6.

### DETAILED DESCRIPTION OF PREFERRED EMBODIMENTS

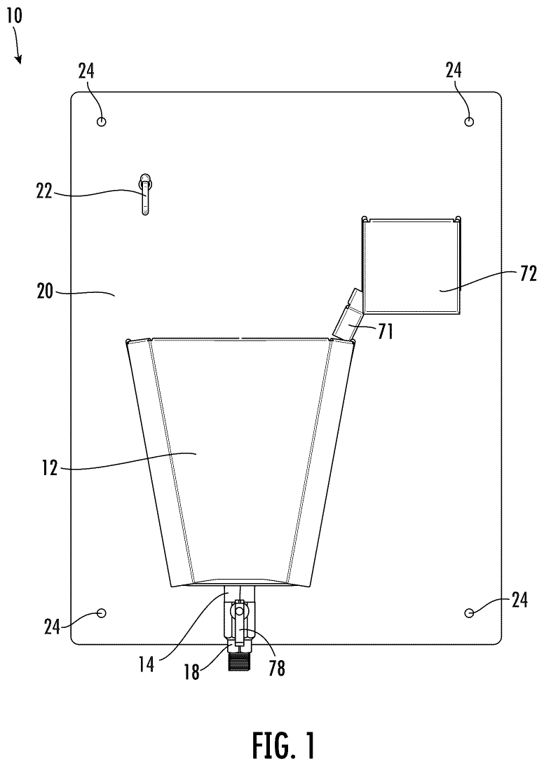

A bedpan washer sink apparatus according to an embodiment of the invention is illustrated in FIGS. 1-5, and shown generally at reference numeral 10. The bedpan washer sink apparatus 10 comprises a container section 12 comprised of opposed side panels and a base panel attached to a substantially flat backing plate 20, and a face panel attached to the opposed side panels. The container section 12 can have the general shape of an inverted truncated pyramid or trapezoidal prism. Alternatively, the container section 12 can comprise a single arcuate sidewall attached to the backing plate 20 and a base. The top of the container section 12 is open, and an opening is formed in the base of the container section 12 for allowing liquid to drain from the container section 12.

Preferably, the bedpan washer sink apparatus 10 is made of 304 stainless steel polished plates that are formed and fusion welded. Preferably, the stainless steel plates are formed and fusion welded to form the bedpan washer sink 10. Preferably, the container section 12 has dimensions of four inches by nine inches by two inches.

A ⅜ inch stainless steel pipe coupling 14 can be welded on the base of the container section 12 around the opening formed in the base. A vertical mounted swing check valve 18 can be connected to the pipe coupling 14, as shown in FIGS. 1-3. The container section 12 can be attached to a backing mounting plate 20. The container section 12 can be stud welded to the backing plate 20 from behind. Preferably, the backing plate 20 is made of 304 stainless steel. Holes 24 can be formed in the backing plate 20. Preferably, four holes 24 are drilled into the backing plate 20, and each hole has a diameter of ¼ inch. A stainless steel hose hook 22 can be positioned on the backing plate 20, as shown in FIGS. 1-3. Additional hose hooks 22 can be positioned on the backing plate 20 if desired. A stainless steel band can be fusion welded around the top of the container 12. The band can have a width of two inches.

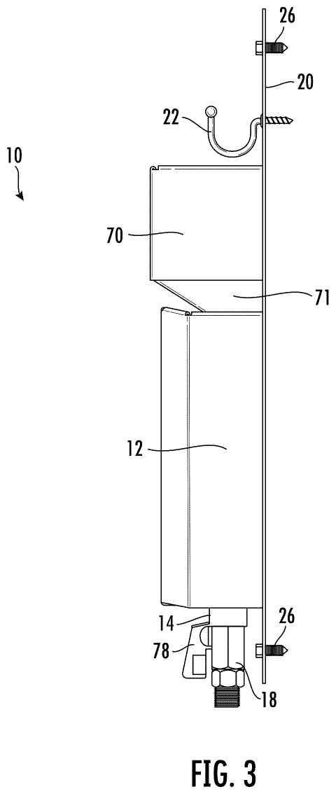

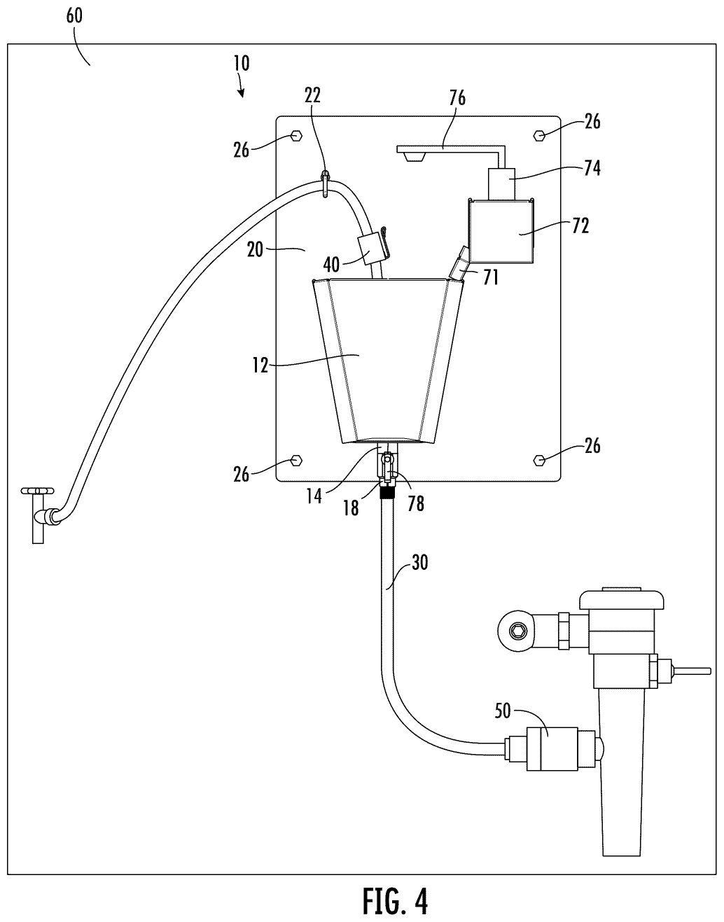

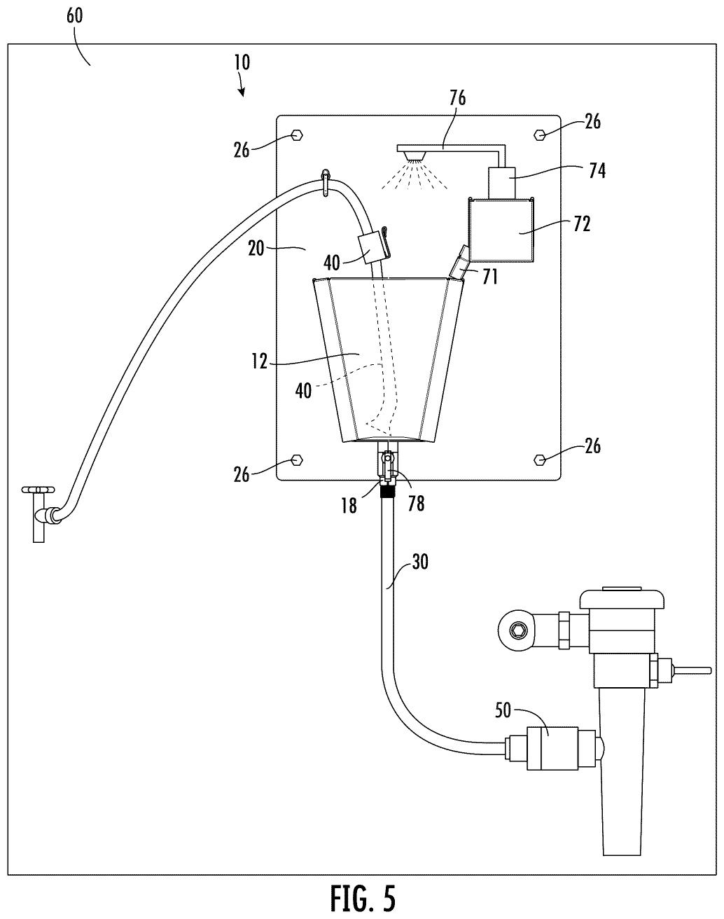

Another embodiment of the invention comprises a method of installing the bedpan washer sink apparatus 10, such as in a room of a hospital, rest home or other care facility. The bedpan sink apparatus 10 can be attached to a wall 60 of the room. Fasteners such as wall screws 26 can be inserted through the holes 24 formed in the back plate 20, as shown in FIG. 3. The wall screws 26 can be screwed into the wall 60 whereby the bedpan sink 10 is attached to the wall 60, as shown in FIGS. 4 and 5. Anchors can also be used with the wall screws 26. The tail piece below the flush valve of a toilet is replaced with a “trap primer” tail piece 50, and a tubular drain line 30 is provided. One end of the drain line 30 can be attached to the vertical mounted swing check valve 18, and the other end of the drain line 30 is attached to the trap primer tail piece 50 below the flush valve, as shown in FIGS. 4 and 5. A bedpan washer wand 40 can be positioned in the container section 12 of the bedpan sink 10, as shown in FIGS. 4 and 5. This method of installing the bedpan washer sink 10 according to an embodiment of the invention is non-invasive.

The bedpan washer wand 40 can be stored in the container section 12 of the bedpan sink 10 when not being used. Any water that leaks from the bedpan washing wand 40 is captured and collected in the container section 12 and drains through the outlet opening at the base of the container section 12, and travels through the drain line 30 to the trap primer tail piece 50 and ultimately a sanitary sewer. As such, the bedpan washer sink apparatus 10 provides a place for storing the bedpan washing wand 40 when not in use and prevents water that is leaking from the bedpan washing wand 40 from contacting the floor, wall and/or other surrounding structures in the room where the bedpan washing wand 40 is located. As such, the bedpan washer sink 10 helps prevent water damage and mold, and promotes a sanitary environment.

Preferably, the bedpan sink apparatus 10 can include an enhanced sanitizing feature that can clean and/or sanitize the washer wand 40, which gets dirty when used to wash out a bedpan. Of course, sanitation and infection control are extremely important in healthcare facilities, in which the bedpan washer sink apparatus 10 is intended to be used.

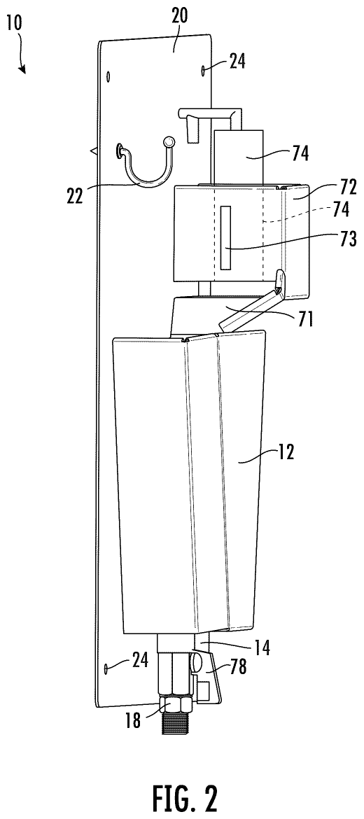

A holding compartment 72 can be positioned on the backing plate 20 above the container section 12, as shown in FIGS. 1-3. The holding compartment 70 can be comprised of a base and two side panels attached to the backing plate 20, and a front panel attached to the two opposed side panels, as shown in FIGS. 1-3. Alternatively the holding compartment can be a single platform, tray or bracket extending outwardly from the backing plate 20. The compartment 72 is intended to support and contain a bottle 74 or other suitable container, as shown in FIGS. 2, 4 and 5. The bottle 74 contains a sanitizing liquid and/or germicidal disinfectant, such as hydrogen peroxide, hypochlorite, paracetic acid, ethyl alcohol, chlorine bleach, antibacterial soap or other suitable material. The bottle 74 includes a pump dispenser 76 for dispensing the sanitizing liquid contained in the bottle 74. A drip chute 71 can be positioned between the compartment 72 and the top of the container section 12, as shown in FIGS. 1 and 2.

According to an embodiment, the bottle 74 can be made of a transparent or translucent material, and an elongate slit opening 73 can be formed in one sidewall of the compartment 72, as shown in FIG. 2, whereby the level of sanitizing liquid contained in the bottle 74 can be viewed and monitored. As such, it is readily apparent to a user when the level of sanitizing liquid is nearing the bottom of the bottle 74 and refilling will soon be needed.

As shown in FIGS. 1-3, a ⅜ inch on/off valve 78 can be positioned below the container section 12 and above the check valve 18. After a user washes a bedpan in the container section 12, the user turns off the on/off valve 78. The washer wand 40 can be used to fill the container section 12 with water if desired. The user can depress the pump 76 of the pump bottle 74 such that liquid sanitizer is dispensed from the pump bottle 74 into the container section 12 of the apparatus 10, as shown in FIG. 5. Any sanitizing liquid leaking from the pump bottle 74 and/or compartment 72 can be caught in the drip chute 71, which channels the liquid into the container section 12. The washer wand 40 is placed in the bath of water and sanitizer in the container section 12. After a desired amount of time, such as about three minutes, the on/off valve 78 is turned on to allow the water and sanitizer to drain into the sanitary sewer. As such, the washing wand 40 and container section 12 are maintained in a sanitary condition. In an alternative embodiment, the pump bottle 74 can be replaced with an automized dispenser for dispensing the liquid sanitizer. For example, the apparatus 10 can comprise an electric liquid pump dispenser having a button that when depressed, the liquid sanitizer is dispensed.

Another embodiment of the invention comprises a plumbing fixture kit comprising the bedpan washer sink 10 and the drain line 30. Another embodiment of the invention comprises a plumbing fixture kit comprising the bedpan washer sink 10, the drain line 30, and the trap primer tail piece 50. A user can purchase the kit and install it in accordance with the method of installation described above. Another embodiment of the invention comprises a kit comprising the bedpan washer sink 10, the drain line 30, the bedpan washing wand 40, and the trap primer tail piece 50. Another embodiment of the invention comprises a kit comprising the bedpan washer sink 10, the drain line 30, and the washing wand 40.

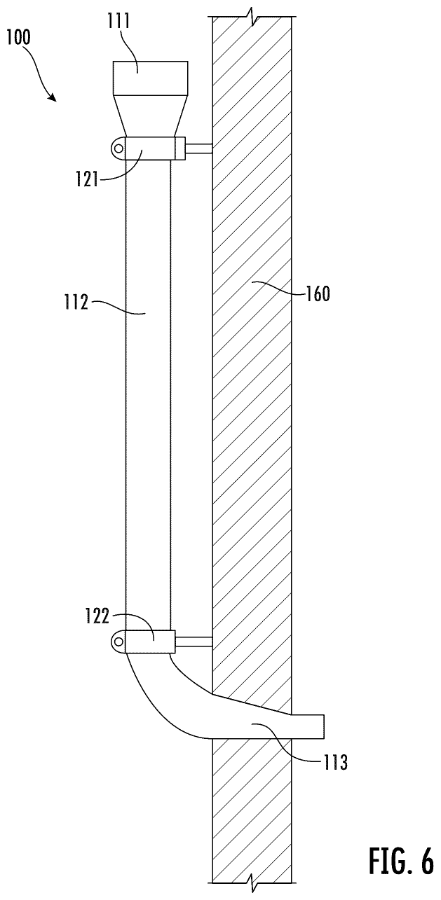

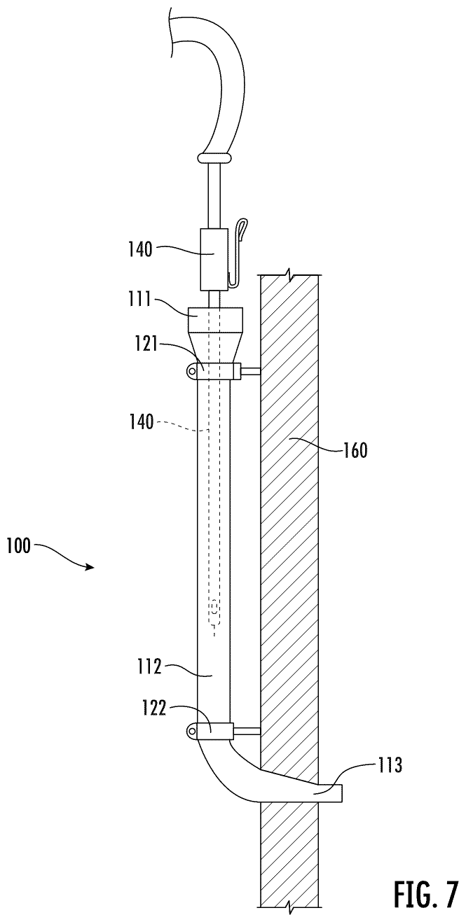

A bedpan washer sink apparatus according to another embodiment of the invention is illustrated in FIGS. 6 and 7, and shown generally at reference numeral 100. The apparatus 100 is comprised of a funnel shaped top opening 111, an elongate cylindrical tube section 112, and an exit spout 113. As shown in FIG. 6, the apparatus 100 can be attached to a wall 160 by collar brackets 121, 122 attached proximate the top and bottom of the tube section 112. A bedpan washing wand 140 can be inserted into the top opening 113 and sit within the tube section 112. Any water that leaks from the bedpan washing wand 140 drains through the base exit spout 113, which can be connected to a drain line that can be connected to a sanitary sewer.

A bedpan washer sink apparatus and methods of installing and using same are described above. Various changes can be made to the invention without departing from its scope. The above description of various embodiments of the invention are provided for the purpose of illustration only and not limitation-the invention being defined by the claims and equivalents thereof.

What is claimed is:

1. A bedpan washer sink apparatus comprising:
(a) a substantially flat backing plate; and
(b) a container section comprising at least one side panel attached to a base panel and the backing plate, the container section defining an interior area for receiving and containing at least a portion of a bedpan washing wand therein, the base panel having an opening formed therein for allowing liquid collected in the container section to drain from the container section.

2. The bedpan washer sink apparatus according to claim 1, wherein the container section comprises two opposed side panels attached to the base panel and the backing plate, and a face panel attached to the two opposed side panels.

3. The bedpan washer sink apparatus according to claim 1, wherein the container section has a shape of a truncated pyramid or a trapezoidal prism.

4. The bedpan washer sink apparatus according to claim 1, wherein a plurality of openings are formed in the backing plate for receiving fasteners therethrough.

5. The bedpan washer sink apparatus according to claim 1, further comprising a pipe coupling attached around the opening formed in the base panel.

6. The bedpan washer sink apparatus according to claim 5, further comprising a check valve connected to the pipe coupling.

7. The bedpan washer sink apparatus according to claim 1, further comprising at least one hook attached to the backing plate adapted to support a hose thereon.

8. The bedpan washer sink apparatus according to claim 1, further comprising a support member attached to the backing plate above the container section for supporting a sanitizing device thereon.

9. The bedpan washer sink apparatus according to claim 8, wherein the support member comprises a compartment comprised of first and second opposed side panels attached to the backing plate, a base panel attached to the first and second opposed side panels and the backing plate, and a face panel attached to the first and second opposed side panels and the base panel.

10. The bedpan washer sink apparatus according to claim 9, further comprising a drip chute positioned between the compartment and the container section.

11. A bedpan washer sink kit comprising:
(a) a bedpan washer sink apparatus comprising a backing plate having a plurality of openings formed therein, and a container section comprising at least one side panel connected to a base panel and the backing plate, the base panel having an opening formed therein for allowing liquid collected in the container section to drain from the container section; and
(b) a plurality of fasteners for positioning through the openings formed in the backing plate and attaching the backing plate to a wall.

12. The kit according to claim 11, wherein the plurality of fasteners comprises wall screws.

13. The kit according to claim 11, wherein the bedpan washer sink apparatus further comprises a pipe coupling attached around the opening formed in the base panel of the container section and a check valve connected to the pipe coupling.

14. The kit according to claim 11, further comprising a tubular drain line having a first end and a second end, the first end of the drain line operatively connected to the check valve.

15. The kit according to claim 14, further comprising a trap primer tail piece operatively connected to the second end of the drain line.

16. The kit according to claim 15, further comprising a bedpan washing wand, wherein at least a portion of the bedpan washing wand is received and contained within the container section.

17. The kit according to claim 16, wherein the bedpan washing wand includes a hose, and the bedpan washer sink apparatus further comprises a hook mounted on the backing plate for supporting the hose thereon.

18. The kit according to claim 16, further comprising a support member mounted on the backing plate above the container section, and a bottle containing a sanitizing liquid, the bottle having a pump dispenser for dispensing the sanitizing liquid from the bottle, the bottle positioned on the support member, wherein the sanitizing liquid exiting the pump dispenser falls into the container section.

19. A method of maintaining a bedpan washer wand in a sanitary condition, comprising the steps of:
(a) providing a bedpan washer sink apparatus comprising a backing plate having a plurality of openings formed therein, and a container section comprising at least one side panel connected to a base panel and the backing plate, the base panel having an opening formed therein for allowing liquid collected in the container section to drain from the container section, an on/off valve operatively connected to the opening formed in the base panel, and a sanitizing device containing a sanitizing liquid;
(b) positioning the backing plate against a wall and positioning fasteners through the openings formed in the backing plate to attaching the backing plate to the wall;
(c) providing a bedpan washer wand for cleaning items, the bedpan washing wand having a terminal end from which liquid is dispensed;
(d) positioning at least a portion of the bedpan washer wand including the terminal end into the container section of the bedpan washer sink apparatus when the bedpan washer wand is not in use;
(e) closing the on/off valve to prevent exit of liquid from the container section;
(f) dispensing sanitizing liquid from the sanitizing device into the container section; and
(g) opening the on/off valve to allow exiting of liquid from the container.

20. The method of claim 19, further comprising the step of operatively connecting a drain line to the bedpan washer sink apparatus and a toilet.
